SEQUENCE LISTING (47 sequences)

SEQ ID NO: 1 - protein, 14 aa, Artificial Sequence
  Description of Artificial Sequence Synthetic peptide
DVFMKGLSMAKEGV

SEQ ID NO: 2 - protein, 13 aa, Artificial Sequence
  Description of Artificial Sequence Synthetic peptide
VFMKGLSMAKEGV

SEQ ID NO: 3 - protein, 12 aa, Artificial Sequence
  Description of Artificial Sequence Synthetic peptide
FMKGLSMAKEGV

SEQ ID NO: 4 - protein, 11 aa, Artificial Sequence
  Description of Artificial Sequence Synthetic peptide
MKGLSMAKEGV

SEQ ID NO: 5 - protein, 10 aa, Artificial Sequence
  Description of Artificial Sequence Synthetic peptide
KGLSMAKEGV

SEQ ID NO: 6 - protein, 9 aa, Artificial Sequence
  Description of Artificial Sequence Synthetic peptide
GLSMAKEGV

SEQ ID NO: 7 - protein, 8 aa, Artificial Sequence
  Description of Artificial Sequence Synthetic peptide
LSMAKEGV

SEQ ID NO: 8 - protein, 7 aa, Artificial Sequence
  Description of Artificial Sequence Synthetic peptide
SMAKEGV

SEQ ID NO: 9 - protein, 6 aa, Artificial Sequence
  Description of Artificial Sequence Synthetic peptide
MAKEGV

SEQ ID NO: 10 - protein, 5 aa, Artificial Sequence
  Description of Artificial Sequence Synthetic peptide
AKEGV

SEQ ID NO: 11 - protein, 4 aa, Artificial Sequence
  Description of Artificial Sequence Synthetic peptide
KEGV

SEQ ID NO: 12 - protein, 14 aa, Artificial Sequence
  Description of Artificial Sequence Synthetic peptide
MDVFMKGLSMAKEG

SEQ ID NO: 13 - protein, 13 aa, Artificial Sequence
  Description of Artificial Sequence Synthetic peptide
MDVFMKGLSMAKE

SEQ ID NO: 14 - protein, 12 aa, Artificial Sequence
  Description of Artificial Sequence Synthetic peptide
MDVFMKGLSMAK

SEQ ID NO: 15 - protein, 11 aa, Artificial Sequence
  Description of Artificial Sequence Synthetic peptide
MDVFMKGLSMA

SEQ ID NO: 16 - protein, 10 aa, Artificial Sequence
  Description of Artificial Sequence Synthetic peptide
MDVFMKGLSM

SEQ ID NO: 17 - protein, 9 aa, Artificial Sequence
  Description of Artificial Sequence Synthetic peptide
MDVFMKGLS

SEQ ID NO: 18 - protein, 8 aa, Artificial Sequence
  Description of Artificial Sequence Synthetic peptide
MDVFMKGL

SEQ ID NO: 19 - protein, 7 aa, Artificial Sequence
  Description of Artificial Sequence Synthetic peptide
MDVFMKG

SEQ ID NO: 20 - protein, 6 aa, Artificial Sequence
  Description of Artificial Sequence Synthetic peptide
MDVFMK

SEQ ID NO: 21 - protein, 5 aa, Artificial Sequence
  Description of Artificial Sequence Synthetic peptide
MDVFM

SEQ ID NO: 22 - protein, 4 aa, Artificial Sequence
  Description of Artificial Sequence Synthetic peptide
MDVF

SEQ ID NO: 23 - protein, 13 aa, Artificial Sequence
  Description of Artificial Sequence Synthetic peptide
DVFMKGLSMAKEG

SEQ ID NO: 24 - protein, 12 aa, Artificial Sequence
  Description of Artificial Sequence Synthetic peptide
DVFMKGLSMAKE

SEQ ID NO: 25 - protein, 11 aa, Artificial Sequence
  Description of Artificial Sequence Synthetic peptide
DVFMKGLSMAK

SEQ ID NO: 26 - protein, 10 aa, Artificial Sequence
  Description of Artificial Sequence Synthetic peptide
DVFMKGLSMA

SEQ ID NO: 27 - protein, 9 aa, Artificial Sequence
  Description of Artificial Sequence Synthetic peptide
DVFMKGLSM

SEQ ID NO: 28 - protein, 8 aa, Artificial Sequence
  Description of Artificial Sequence Synthetic peptide
DVFMKGLS

SEQ ID NO: 29 - protein, 7 aa, Artificial Sequence
  Description of Artificial Sequence Synthetic peptide
DVFMKGL

SEQ ID NO: 30 - protein, 6 aa, Artificial Sequence
  Description of Artificial Sequence Synthetic peptide
DVFMKG

SEQ ID NO: 31 - protein, 5 aa, Artificial Sequence
  Description of Artificial Sequence Synthetic peptide
DVFMK

SEQ ID NO: 32 - protein, 4 aa, Artificial Sequence
  Description of Artificial Sequence Synthetic peptide
DVFM

SEQ ID NO: 33 - protein, 3 aa, Artificial Sequence
  Description of Artificial Sequence Synthetic peptide
DVF

SEQ ID NO: 34 - protein, 8 aa, Artificial Sequence
  Description of Artificial Sequence Synthetic peptide
GLSMAKEG

SEQ ID NO: 35 - protein, 7 aa, Artificial Sequence
  Description of Artificial Sequence Synthetic peptide
GLSMAKE

SEQ ID NO: 36 - protein, 6 aa, Artificial Sequence
  Description of Artificial Sequence Synthetic peptide
GLSMAK

SEQ ID NO: 37 - protein, 5 aa, Artificial Sequence
  Description of Artificial Sequence Synthetic peptide
GLSMA

SEQ ID NO: 38 - protein, 4 aa, Artificial Sequence
  Description of Artificial Sequence Synthetic peptide
GLSM

SEQ ID NO: 39 - protein, 3 aa, Artificial Sequence
  Description of Artificial Sequence Synthetic peptide
GLS

SEQ ID NO: 40 - protein, 2 aa, Artificial Sequence
  Description of Artificial Sequence Synthetic peptide
GL

SEQ ID NO: 41 - protein, 7 aa, Artificial Sequence
  Description of Artificial Sequence Synthetic peptide
LSMAKEG

SEQ ID NO: 42 - protein, 6 aa, Artificial Sequence
  Description of Artificial Sequence Synthetic peptide
LSMAKE

SEQ ID NO: 43 - protein, 5 aa, Artificial Sequence
  Description of Artificial Sequence Synthetic peptide
LSMAK

SEQ ID NO: 44 - protein, 4 aa, Artificial Sequence
  Description of Artificial Sequence Synthetic peptide
LSMA

SEQ ID NO: 45 - protein, 3 aa, Artificial Sequence
  Description of Artificial Sequence Synthetic peptide
LSM

SEQ ID NO: 46 - protein, 2 aa, Artificial Sequence
  Description of Artificial Sequence Synthetic peptide
LS

SEQ ID NO: 47 - protein, 15 aa, Artificial Sequence
  Description of Artificial Sequence Synthetic peptide
MDVFMKGLSMAKEGV

(19) United States
(12) Patent Application Publication
(10) Pub. No.: US 2006/0036073 A1
(43) Pub. Date: Feb. 16, 2006

(54) Neurotrophic and neuroprotective peptides

(76) Inventor: Manfred Windisch, Graz (AT)

Correspondence Address: YOUNG & THOMPSON, 745 SOUTH 23RD STREET, 2ND FLOOR, ARLINGTON, VA, 22202, US

(21) Appl. No.: 10/509,095

(22) PCT Filed: Mar. 10, 2003
(86) PCT No.: PCT/AT03/00065

§ 371 Date: Apr. 13, 2005

(30) Foreign Application Priority Data
Mar. 28, 2002 (AT) A 495/2002

(51) Int. Cl. C07K 7/08; C07K 7/06

(52) U.S. Cl. 530/326; 530/327; 530/328; 530/329; 530/330

(57) ABSTRACT

Novel peptides, whose individual components are L-amino acids or D-amino acids are used as active ingredients in medicaments for treating diseases in which the increased occurrence of free radicals plays a pathophysiological role, or for treating diseases involving acute hypoxia or ischaemia in an organ system of the body, in particular in the central nervous system, or for treating iron-storage diseases such as Hallervorden-Spatz syndrome, or for treating neurodegenerative diseases, in particular Alzheimer's disease, the Lewy body variant of Alzheimer's disease, Parkinson's disease, multi-system atrophy, Lewy body dementia or Huntington's chorea and all syndromes that are similar to the neurodegenerative diseases.

### TECHNICAL FIELD

This invention relates to peptides that are 4 to 14 amino acids in length. The peptides according to the invention can be used as active ingredients in pharmaceutical agents for treating degenerative diseases of the central nervous system, such as Alzheimer's disease, Lewy Body dementia, Parkinson's disease, Huntington's disease (chorea), multisystem atrophy and other similar diseases.

Prior Art

In neurodegenerative diseases, aggregates of proteins in the brain generally occur as a common feature. In the case of Alzheimer's disease, the so-called senile plaques are extracellular albumin deposits that first and foremost consist of amyloid-beta peptides and the so-called neurofibrillary tangles, intracellular protein glomeruli from hyperphosphorylated tau protein. With Parkinson's disease, intracellular inclusion bodies, consisting of aggregated alpha-synuclein, are found. According to the most recent scientific findings, it was possible to detect such inclusion bodies, namely Lewy Bodies, in more than 70% of patients suffering from familial and sporadic Alzheimer's disease; they are also found in patients who suffer from Down's syndrome. Aggregates of alpha-synuclein in glia cells occur in the multisystem atrophy. Analogously to this, aggregates of prion protein are found in Creutzfeldt-Jakob disease and related diseases, and ultimately Huntington deposits in Huntington's chorea.

In many of the patients, mutated proteins are present that have an especially pronounced aggregation behavior. In the majority of patients, however, the aggregates consist of normal wild-type proteins. Various factors that suddenly change the solubility behavior of the proteins are assumed, whereby, for example, increased oxidative stress during the aging process should play an essential role. Even changes in the capacity of various protein-decomposing enzymes are also suitable as factors, since albumins that are improperly modified by disorders can result, which are then deposited and can no longer be further processed by various disposal enzymes.

Another triggering pathophysiological mechanism consists in a disrupted equilibrium between aggregatory and anti-aggregatory proteins. The discovery that the synaptic protein alpha-synuclein represents the main component of the so-called Lewy Bodies and that mutations in this protein lead to the familial Parkinson's disease have made this albumin the focal point of scientific research. In addition to alpha-synuclein, gamma-synuclein and beta-synuclein as well as the recently discovered synoretin exist as additional representatives of this protein family (Surgurchov et al., Mol. Cell. Neurosci. 13(2): 95-103 [1999]).

In the case of various neurodegenerative diseases, an alteration of the quantitative ratio between the individual synucleins occurs to the extent that the relative proportion of alpha-synuclein is increased. It was possible to detect in vitro that beta-synuclein, a very close relative of alpha-synuclein, is able to inhibit the aggregation of alpha-synuclein in a dose-dependent manner (Hashimoto et al., Neuron 32 (2): 213-23 [2001]). Tests in cell cultures in which a disruption of the normal cell proliferation and differentiation was triggered by over-expression of alpha-synuclein also showed an advantageous action, in the therapeutic sense, of beta-synuclein, which further normalized the adhesion, survival and growth of neurites in these cultures. Mice, which are transgenic for alpha-synuclein, show an elevated production of this albumin and therefore exhibit a disrupted ratio in the amounts between alpha- and beta-synuclein. Over the course of aging, they form intraneuronal inclusion bodies that are similar to Lewy Bodies and also show progressive motor disruptions, which are comparable to the disruption of function in Parkinson's disease. If these animals with beta-synuclein are crossed with transgenic animals, which show an elevated expression of this albumin, a significantly higher level in the overall expression of the synucleins can restore a homoeostasis. As a result, the number of inclusion bodies is highly significantly reduced, and the characteristic neuronal function loss is completely prevented.

Alpha-synuclein should also play an especially important role in the pathology of Alzheimer's disease, however. This is also indicated by the fact that a portion of this protein, the NACP (Non-Amyloid Component Protein) domain, could be demonstrated as part of the senile plaques (Yoshimoto et al., Proc. Natl. Acad Sci 92, 9141-5 [1995] and WO-9506407), and in addition the fact that—as mentioned above—about 70% of patients suffering from Alzheimer's disease exhibit Lewy Bodies in various areas of the brain, in which alpha-synuclein is also found (Eizo et al., Neurosci. Lett. 290 (1), 41-4 [2000]). In a transgenic mouse model, beta-amyloid increases the accumulation and the neurotoxicity of alpha-synuclein (Masliah et al., Proc. Natl. Acad. Sci 98 (21): 12245-50 [2001]). In addition, it was possible for alpha-synuclein, as a synaptic protein, to play an important role in the initial synaptic degeneration and thus to occupy a key role in pathogenesis.

At this time, no causally effective therapies whatsoever of Alzheimer's disease, Lewy Body dementia, Parkinson's disease, or other neurodegenerative diseases are available. A prevention of abnormal protein aggregation by an endogenic factor thus could represent a first step in this direction. Since alpha-synuclein and beta-synuclein interact in addition with various endogenic signal transduction cascades, such as protein kinase C or phospholipase D2 and various transcription factors, other positive influences, which could have a neuroprotective effect, were conceivable with these modes of action.

The use of beta-synuclein and in particular peptides derived therefrom in connection with alpha-synuclein is known; see, for example, octapeptides according to WO-A-02/04482 and three additional peptides in WO-A-02/04625. WO-A-002/0020 and WO-A-01/60794 describe the use of beta-synuclein as a whole molecule or methods that increase its expression in vivo for therapy of neurological diseases that are associated with alpha-synuclein. WO-A-01/60794 in particular also teaches the use of a peptide with the amino acid sequence MDVFMKGLSMAKEGV, which corresponds to the N-terminal amino acids 1 to 15 of the beta-synuclein, for preventing the binding of alpha-synuclein and beta-amyloid. WO-A-01/60794, however, does not yield any evidence of an actual protective action of this peptide on living, neuronal cells and does not contain any references to other active peptides in this sequence range. Shorter peptides were very advantageous for use as pharmaceutical agents, however, since in general with decreasing chain length, the problems of chemical and biological stability as well as bioavailability are greatly reduced.

Presentation of the Invention

The object of this invention is to avoid the drawbacks that are known from the prior art. According to the invention, peptides are proposed that are selected from the group

```
DVFMKGLSMAKEGV
VFMKGLSMAKEGV
FMKGLSMAKEGV
MKGLSMAKEGV
KGLSMAKEGV
GLSMAKEGV
LSMAKEGV
SMAKEGV
MAKEGV
AKEGV
KEGV
                  MDVFMKGLSMAKEG
                  MDVFMKGLSMAKE
                  MDVFMXGLSMAK
                  MDVFMKGLSMA
                  MDVFMKGLSM
                  MDVFMXGLS
                  MDVFMKGL
                  MDVFMKG
                  MDVFMK
                  MDVFM
                  MDVF
                  DVFMKGLSMAKEG
                  DVFMKGLSMAKE
                  DVFMKGLSMAK
                  DVFMKGLSMA
                  DVFMKGLSM
                  DVFMKGLS
                  DVFMKGL
                  DVPMKG
                  DVFMK
                  DVFM
                  DVF
                  GLSMAKEG
                  GLSMAKE
                  GLSMAK
                  GLSMA
                  GLSM
                  GLS
                  GL
                  LSMAKEG
                  LSMAKE
                  LSMAK
                  LSMA
                  LSM
                  LS
```

These peptides according to the invention are derived from the N-terminal sequence of the beta-synuclein and antagonize the influence of toxic or vitality-damaging noxae, as they exist in neurodegenerative diseases.

Surprisingly enough, it turned out, as could not have been derived from the prior art, that even individual peptides, which comprise only half or even only one-third of the sequence of 15 amino acids that is described in WO-0160794, in models of pathological processes, as they are present or expected in neurodegenerative diseases, exert excellent action; for example the heptapeptide SMAKEGV and the pentapeptide LSMAK.

Not only peptides whose individual components are L-amino acids, but also peptides whose individual components are D-amino acids, are within the scope of the invention.

Also within the scope of the invention, N- or C-terminally altered peptides are considered.

Other advantageous embodiments of the peptides according to the invention are disclosed in the subclaims.

In addition, the invention relates to pharmaceutical agents that contain the peptides according to the invention as pharmaceutical active ingredients.

The peptides of the invention can be synthetically produced in various ways.

The chemical synthesis of a peptide represents a conventional process and can be achieved by, for example, the Merrifield Solid-Phase Synthesis Technique (Merrifield, J., Am. Chem. Soc., 85:2149-2154 [1963]; Kent et al., Synthetic Peptides in Biology and Medicine, 29 ff eds. Alitalo et al., Elsevier Science Publishers 1985; Haug, J. D. Peptide Synthesis and the Protecting Group Strategy, American Biotechnology Laboratory, 5 (1): 40-47 [1987]). Processes of chemical peptide synthesis also involve the use of automatic peptide synthesizers with use of commercially available protected amino acids, such as, for example, Biosearch (Models 9500 and 9600), Applied Biosystems Inc. (Model 430); Miligen (Model 9050), etc. In addition to the chemical processes, these peptides can be produced by means of recombinant technology in the cells of bacteria, fungi or mammals and can be purified by means of conventional processes.

Independently of whether the synthesis of the peptides according to the invention is performed with chemical methods or by recombinant technologies, it may be desirable to modify the peptides in particular to increase the stability after introduction into the organism to be treated. To this end, for example, the following methods can be used:

Advantageous Methods for Implementing the Invention

1) Covalent modifications, in which predetermined amino acid radicals of the peptide can react with organic derivatization substances on selected side chains or terminal radicals. For example, cysteinyl radicals react with alpha-haloacetates and corresponding amines, such as chloroacetic acid or chloroacetamide, and in this case produce carboxymethyl or carboxyamidomethyl derivatives. Cysteinyl radicals can also be derivatized by the reaction with bromotrifluoroacetone, alpha-bromo-beta (5-imidozoyl)propionic acid, chloroacetyl-phosphate, N-alkylmalemides, 3-nitro2-pyridyldisulfide, methyl-2-pyridyldisulfide, p-chloromercuric benzoate, 2-chloromercuric-4-nitrophenol or chloro-7-nitrobenzo-2-oxa-1,3-diazole. The amino acid histidine can also easily be derivatized by the reaction with diethyl procarbonate at a pH of 5.5-7, since this substance is relatively specific to the histidyl side chain. Parabromophenazyl bromide is also a possibility, whereby the reaction is preferably implemented in 0.1 molar sodium cacodylate at pH 6.0.

2) Lysine and amino-terminal radicals can also be derivatized with succinate or other carboxylic acid anhydrides. The reaction with these agents has the effect of reversing the charge of the lysinyl radical. Other suitable reagents for the derivatization of radicals that contain alpha-amino include imido-esters, such as methyl bicolinimidate, pyridoxal-phosphate, pyridoxal, chloroborohydride, trinitrobenzenesulfonic acid, O-methyl isourea, 2,4 pentanedione, and transaminase-catalyzed reactions with glyoxylate. The arginyl radicals can be modified by the reaction with one or more conventional reagents, such as phenyl glyoxal, 2,3 butanedione, 1,2-cyclohexanedione and ninhydrin. The derivatization of the arginyl radicals requires that the reaction be performed under alkaline conditions because of the high PK value of the guanidine group. In addition, these reagents can also react with groups of lysine, as well as with the arginine-epsilon amino group.

3) Tyrosyl radicals are known targets for the introduction of spectral labelings by the reaction with aromatic diazonium substances or tetranitromethane. N-Acetylimidizole and tetranitromethane are most frequently used to produce O-acetyltyrosyl and 3-nitro derivatives.

4) The carboxyl side group (aspartyl or glutamyl) is modified selectively by reaction with carbodiimides (R′—N—C—N—R′) such as 1-cyclohexyl-3-(2-morpholinyl (4-ethyl)) carbodiimide or 1-ethyl-3-(4-azonia-4,4-diethylpentyl)-carbodiimide. Aspartyl and glutamyl radicals are converted into asparaginyl and glutaminyl radicals by the reaction with ammonium ions. Glutaminyl and asparaginyl radicals are frequently deamidated to the corresponding glutamyl and aspartyl radicals.

5) Other modifications contain the hydroxylation of proline and lysine, the phosphorylation of the hydroxyl groups of seryl and threonyl radicals, the methylation of the alpha-amino group of lysine, argenine and histidine side chains, as well as the acetylation of the N-terminal amino group and the amidation of the C-terminal carboxyl groups.

Such derivatizations can be used to improve the solubility, absorption, the biological half-life and the like. Alternatively, the derivatizations can also be used to minimize some undesirable side effects of the proteins.

Determination of Biological Activity:

Since the targets of the therapy with the peptides presented according to the invention are various neurodegenerative diseases, different model systems for detecting the biological activity of the peptides according to the invention in the case of neurodegenerative diseases were used. Later, the individually used model systems and the results thus achieved are described.

It is common to these biological test systems that cortical neurons that are obtained from chicken embryos are cultivated in culture plates for eight days and then are exposed to a specific noxa (Pettmann et al., Nature 281 (5730): 378-80 [1979]).

To this end, a one-day-old, fertilized hen's egg is incubated for eight days at +12 +/− 0.1° C. and 80 +/− 5% atmospheric humidity. On the embryonal day 0, the eggs are transferred into an incubator and incubated up to the embryonal day 8 at 38 +/− 0.5° C. and 55 +/− 5%. After the brains are removed, the cortices are isolated, homogenized, and neurons are taken into primary culture (culture conditions: Dulbecco's Modified Eagle's Medium, 20% v/v fetal calf serum, 0.01% gentamicin, 1 g/l of glucose, 2 mmol of L-glutamine, +37° C., 5% CO₂ and 95% atmospheric humidity). After 8 days in the culture, the peptide to be examined is added (final concentrations of 1.56 to 200 μm) and the specified noxa is removed. In each test, the result is a damaged control and a vehicle control. After the end of the specified stress period, the proportion of the still living neurons is determined with a metabolic colorimetric assay (the reaction of the yellow chromophore MTT to a blue formazan product is carried out only by living cells).

See Table 1 in connection with the amino acid sequences of the cited peptides.

### EXAMPLE 1

### Serum-Withdrawal Assay

By the withdrawal of growth factors (reduction of the admixing of fetal calf serum to 2% v/v), a slow and progressive cell death by apoptosis and neurodegeneration is created. This simulates the disruptions in the stimulation of neurons with nerve growth factors that are assumed to be one of the possible causes of neurodegenerative diseases. The effectiveness of the new peptides in preventing cell death was measured.

As a whole, in this test, 31 of 45 tested peptide fragments had neuroprotective, anti-apoptotic potential. In this case, substances BH#16 and BH#37, whose effects were 150% over the effects of the control (=100%), were especially efficient. In the case of octapeptide BH#7 (LSMAKEGV sequence), the effects were approximately 450%.

### EXAMPLE 2

### Chronic Disruption of the Calcium Metabolism by Ionomycin

It is expected that in the case of various neurodegenerative diseases, chronic calcium overloading occurs because of metabolic malfunctions, which ultimately produces cell death via the activation of various enzyme systems. In this model, this damage is induced over 24 hours by an addition of ionomycin in methanolic solution (final concentration: 10 μM). Methanol, diluted in a medium, is used as a vehicle control.

In this ischemic damage model, 6 substances were neuroprotectively active. Primarily 3 substances are advantageous: BH#8, BH#13 and BH#34 resulted in an increase in cell vitality to about 150%.

### EXAMPLE 3

### Oxidative Stress by Iron Chloride

Long-lasting treatment with iron chloride represents a chronic oxidative stress that causes nerve cells, but also other cells, to become necrotic. Since disruptions in the iron balance are described both for Alzheimer's disease and for Parkinson's disease, but especially in the case of Recklinghausen-Appelbaum diseases, such as the Hallervorden-Spatz disease, this model represents a relevant test system. On the 8^{th} culture day, nerve cells are damaged by adding 10 μl of FeCl2 solution (final concentration: 1 mmol). Damage is done for 24 hours.

In this assay, 23 peptide fragments exhibited a definitive neuroprotective action, and an increase in the vitality to over 150% was present in more than one-half of the substances. More substances than in any other damage assay resulted in a cell vitality increase of over 200% (BH#8, BH#10, BH#11, BH#13) or 250% (BH#15, BH#6, BH#27, BH#28).

### EXAMPLE 4

### Oxidative Stress By Hydrogen Superoxide

By the addition of hydrogen superoxide to the culture medium, free radicals are produced that bring about massive cell death in nerve cell cultures. Since this represents a ubiquitous mechanism of cell damage, this model has relevance both for acute and for chronic nerve degeneration. On the 8^{th} culture day, H₂O₂ is added to the nerve cell cultures to a final concentration of 100 μm.

A total of 30 peptides showed neuroprotective potential here. With effects of over 200% compared to the damaged control (=100%), the substances BH#5, BH#8, BH#9 and BH#29 were especially significant. The peptides BH#13, BH#29, BH#37, BH#38 and BH#46 also showed a neuronal vitality increase to 145% and more.

### EXAMPLE 5

### Amyloid-Beta Aggregation Assay

Beta-Amyloid peptides represent, in aggregated form, a potent neurotoxin whose addition to the nerve cell cultures results in a quick and progressive cell death. Since beta-amyloid peptides constitute an essential role in the pathogenesis of Alzheimer's disease, this model can be considered especially relevant.

This biological test is a process for testing anti-aggregatory substance potential that is specially developed for this project. The newly synthesized peptides are added directly to a fresh solution of amyloid-beta peptides to prevent the formation of neurotoxic aggregates. The effect that aggregates that still develop have on growth and survival of nerve cell cultures is the measurement parameter.

Six of the tested 45 peptide fragments could partially compensate for the neurotoxic action of β-amyloid 25-35. The peptides BH#24 and BH#26, which result in an increase of neuronal vitality by 44 or 74%, are significant. However, it was possible to test other peptides, whose synthesis was difficult or since only a little material was available, only once. It therefore cannot be ruled out that one or the other peptide also could be effective.

### EXAMPLE 6

### Cytotoxic Action of Pre-Aggregated Beta-Amyloid Peptide

In contrast to the above-described test, the latter operates with pre-formed neurotoxic amyloid aggregates. To produce the latter, a beta-amyloid peptide (B-A₍₂₅₋₃₅₎), consisting of the amino acids 25 to 35, is dissolved in phosphate-buffered common salt (1 mmol) and stored for at least 72 hours for complexing at room temperature. On the 8^{th} culture day, this solution is pipetted into the culture in a final concentration of 20 μm and as usual the proportion of living cells is determined after 24 hours of exposure.

21 of the 45 peptides examined showed neuroprotective potential. The effects were between 120 and 150% compared to the undamaged vehicle control, whereby partial effects can already be detected in very low dosages.

Except for the aggregation assay (Example No. 5), for which only one round of the testing took place, the mean value (MW) and the standard deviation (Stabw) of at least two independent experiments (n>6) are shown in all figures. That is to say, the implementation of the tests was carried out on different days with different cell preparations and by different individuals. For some peptides, two numbers were issued (#1=#23 or #6=#35 or #7=#43), but the results with these peptides were only shown once. It was not possible to test a peptide, since it could not be brought into solution (#49). With some other substances, there were problems in the production, and thus only small amounts were available, and it was not possible to test the substances in all the assays (#12, 14, 17, 35 and #48). In general, only a few peptides were not effective in any assay (, 18, , 32, , 41, ). The peptides with numbers #12, 20, 30, 33, 39 and 45 were effective in only one screening assay. As could be seen from the figures, the standard deviations, i.e., the fluctuations between the independent experiments, are to some extent quite large. These fluctuations can be attributed to stability problems.

TABLE 1
Amino Acid Sequences of the Tested Beta-Synuclein Peptides and Results Thereof in
the Biological Test Systems of Examples 1 to 6

| | | | | | | | | |
|---|---|---|---|---|---|---|---|---|
| | | | 2% Assay (% | Ionomycin (% | FeCl2 (% | H2O2 (% | β-Amyl (% | AβP reagg. |
| Code | AA-Sequence | # AA | viability) | viability) | viability) | viability) | (% viability) | (diff vs C) |

| | | | | | | | | |
|---|---|---|---|---|---|---|---|---|
| BH#1 | DVFMKGLSMAKEGV | 14 | 132 ± 28.8 | | 125 ± 51.3 | | | 23% |
| BH#2 | VFMKGLSMAKEGV | 13 | | 133 ± 6.2 | 184 ± 119.5 | 140 ± 33.2 | | |
| BH#3 | FMKGLSMAKEGV | 12 | 146 ± 59.2 | | 144 ± 77.7 | | | |
| BH#4 | MKGLSMAKEGV | 11 | 155 ± 16.2 | | 146 ± 31.9 | | 151 ± 31.1 | |
| BH#5 | KGLSMAKEGV | 10 | 143 ± 52.4 | 137 ± 18.4 | 145 ± 70.0 | | | |
| BH#6 | GLSMAKEGV | 9 | | | 153 ± 71.5 | 130 ± 30.0 | 120 ± 11.2 | |
| BH#7 | LSMAKEGV | 8 | | | 183 ± 113.7 | 122 ± 22.9 | 124 ± 12.3 | |
| BH#8 | SMAKEGV | 7 | 139 ± 29.6 | 156 ± 19.4 | | | 23% | |
| BH#9 | MAKEGV | 6 | | | 123 ± 81.0 | | | |
| BK#10 | AKEGV | 5 | 139 ± 34.8 | | | | | |
| BH#11 | KEGV | 4 | | | | 132 ± 50.0 | 131 ± 11.4 | |
| BH#12 | MDVFMKGLSMAKEG | 14 | 156 ± 82.9 | X | | | | |
| BH#13 | MDVFMKGLSMAKE | 13 | 166 ± 11.1 | | | 151 ± 54.2 | 129 ± 14.0 | |
| BH#14 | MDVFMKGLSMAK | 12 | 122 ± 26.9 | X | X | X | X | |
| BH#15 | MDVFMKGLSMA | 11 | 149 ± 49.8 | | | 126 ± 28.0 | | |
| BH#16 | MDVFMKGLSM | 10 | | | | 144 ± 20.4 | 132 ± 44.6 | |
| BH#17 | MDVFMKGLS | 9 | 169 ± 103.1 | 124 ± 7.3 | 150 ± 116.8 | X | 144 ± 26.7 | |
| BH#18 | MDVFMKGL | 8 | | | | | | |
| BH#19 | MDVFMKG | 7 | | | 131 ± 33.6 | 127 ± 32.1 | 132 ± 14.9 | 20% |
| BH#20 | MDVFMK | 6 | | | 132 ± 36.7 | | | |
| BH#21 | MDVFM | 5 | 151 ± 68.8 | | | 126 ± 10.3 | 137 ± 33.0 | |
| BH#22 | MDVF | 4 | 169 ± 103.1 | | 140 ± 36.0 | | 120 ± 10.8 | |
| BH#24 | DVFMKGLSMAKEG | 13 | 125 ± 19.0 | | | | 122 ± 3.0 | |
| BH#25 | DVFMKGLSMAKE | 12 | 169 ± 43.8 | | | | 149 ± 21.0 | |
| BH#26 | DVFMKGLSMAK | 11 | | | | | | |
| BH#27 | DVFMKGLSMA | 10 | | | | 147 ± 7.9 | 128 ± 14.6 | |
| BH#28 | DVFMKGLSM | 9 | | | | 130 ± 77.3 | 144 ± 8.6 | |
| BH#29 | DVFMKGLS | 8 | | | 157 ± 22.1 | 154 ± 42.7 | | |
| BH#30 | DVFMKGL | 7 | | | | | 126 ± 32.0 | |
| BH#31 | DVFMKG | 6 | | | 138 ± 47.5 | 124 ± 37.6 | 143 ± 55.9 | |
| BH#32 | DVFMK | 5 | | | | | | |
| BH#33 | DVFM | 4 | | | | | 145 ± 20.9 | |
| BH#34 | DVF | 3 | | | | | 136 ± 13.4 | 19% |
| BH#36 | GLSMAKEG | 8 | 183 ± 14.9 | | 125 ± 26.6 | | 134 ± 23.9 | |
| BH#37 | GLSMAKE | 7 | | | | 144 ± 32.5 | | |
| BH#38 | GLSMAK | 6 | 172 ± 74.4 | | 152 ± 72.8 | 147 ± 14.3 | | |
| BH#39 | GLSMA | 5 | | | | | | |
| BH#40 | GLSM | 4 | 164 ± 72.5 | | 144 ± 30.4 | | | |
| BH#41 | GLS | 3 | | | | | | |
| BH#42 | GL | 2 | | 120.0 ± 16.4 | | | 128 ± 18.4 | |
| BH#44 | LSMAKEG | 7 | 183 ± 97.1 | 121.0 ± 28.0 | | | | |
| BH#45 | LSMAKE | 6 | 148 ± 53.4 | | | | | |
| BH#46 | LSMAK | 5 | | | | 147 ± 22.0 | | |
| BH#47 | LSMA | 4 | 135 ± 7.5 | | | | | |
| BH#48 | LSM | 3 | | 120 ± 15.4 | | | 120 ± 26.5 | |

Table 2 below summarizes the characteristics of the most important substances from this screening. These peptides are the peptides that are preferred within the scope of the invention.

TABLE 2
Summarized Presentation of the Most Significant Beta-Synuclein
Peptides with Respect to Their Neuroprotective Action

| Code | AA Sequence: | AA | Active in: | Comments |
|---|---|---|---|---|
| BH#8 | SMAKEGV | 7 | 5 of 7 | Small peptide, ex- |
| | | | assays | tremely high effects, |
| | | | | active in a great |
| | | | | number of assays, |
| | | | | therapeutically |
| | | | | advantageous |
| BH#13 | MDVFMKGLSMAKE | 13 | 5 of 7 | Active in a great |
| | | | assays | number of assays |
| BH#16 | MDVFMKGLSM | 10 | 4 of 7 | Active in a great |
| | | | assays | number of assays |
| BH#26 | DVFMKGLSMAK | 11 | 2 of 7 | Especially advan- |
| | | | assays | tageous as a group, |
| | | | | since very similar, |
| BH#27 | DVFMKGLSMA | 10 | 4 of 7 | very high effects |
| | | | assays | from the amino acid |
| | | | | sequence |
| BH#28 | DVFMKGLSM | 9 | 4 of 7 | |
| | | | assays | |
| BH#46 | LSMAK | 5 | 2 of 7 | High effects, |
| | | | assays | smallest peptide, |
| | | | | therapeutically |
| | | | | advantageous |

In the Description, the claims, and in Tables 1 and 2:
A stands for D- or L-alanine,
D stands for D- or L-asparaginic acid,
E stands for D- or L-glutaminic acid,
F stands for D- or L-phenylalanine,
G stands for D- or L-glycine,
K stands for D- or L-lysine,
L stands for D- or L-leucine,
M stands for D- or L-methonine,
S stands for D- or L-serine, and
V stands for D- or L-valine.
   Dosages and Forms of Administration:

In general, the compounds according to the invention are administered in therapeutically effective amounts in pharmaceutically acceptable vehicles or solvents. Such vehicles include (but are not limited to) physiological common salt solution, buffered common salt solution, dextrose, water, glycerol, ethanol and combinations made therefrom. The respective formulation is to be matched to the type of administration.

If necessary, the composition can also contain different amounts of moisture donors or emulsifiers or pH-buffered substances. The pharmaceutical composition can be a liquid solution, a suspension, an emulsion, a tablet, a pill, a capsule, a timed-release formulation or a powder. The preparation can also be produced as a suppository with traditional binding agents and vehicles such as triglycerides. Oral formulations can contain standard vehicles such as mannitol, lactose, starch, magnesium stearate, sodium saccharine, cellulose, magnesium carbonate, etc., in a pharmaceutical degree of purity. Various administration systems are known and can be used to ensure the therapeutic use of the substances according to the invention, such as, e.g., encapsulation in liposomes, microparticles, microcapsules, i.a.

The form of administration is prepared in accordance with a routine process as a pharmaceutical form of administration that is adapted for intravenous administration in humans or other mammals. Typically, the compositions for the intravenous administration are solutions in sterile isotonic aqueous buffer solution. If necessary, the preparation can also contain solubilizers and locally active anesthetics to alleviate the pain at the injection site.

In general, the components are made available either separately or mixed in a dosage unit, for example as a dry freeze-dried powder or anhydrous concentrate in a hermetically sealed container, such as an ampoule, on which the amount of the active pharmaceutical agent is indicated.

When the dispensing form has to be administered as an infusion, it can be dissolved in an infusion flask that contains sterile water or salt solution in a pharmaceutical degree of purity. If the preparation is always administered by injection, an ampoule with sterile water for injection purposes or common salt solution can be made available, such that the individual components can be mixed according to directions before administration.

The therapeutic substances, which are described in the invention, can be formulated both as a neutral form and as a salt. Pharmaceutically acceptable salts include those that were formed with the free amino groups, e.g., those that originate from the hydrochloric acid or the oxalic acid and those that are formed with free carboxyl groups, such as those derived from sodium, potassium, ammonium, calcium, iron oxides, isopropylamine, triethylamine, 2-(ethylamino)ethanol, histidine, procaine, etc.

The amount of the therapeutic agent, which is described in the invention, must be effective for the treatment of the special disease or the condition, is dependent on the nature of the disease or the condition, and is determined by standardized clinical processes. The exact dose, which must be used in the invention, also depends on the type of administration and the degree of severity of the disease or the disorder, and this amount should be adapted with allowance for the specific circumstances of the patient, based on the attending physician's assessment. Suitable dosage ranges for intravenous administration in general are between 20-4,000 μg of the active component per kg of body weight. Suitable dosages for intranasal applications are in the range of between 0.01 mg per kg of body weight up to 1 mg per kg of body weight. Effective dosages for oral applications are in the range of 1 mg to 1,000 mg per kg of body weight and per day. The effective dosages are extrapolated from dose-response curves, which are derived from in vitro models or animal model test systems.

1. A peptide comprising an amino acid sequence selected from the group that consists of

| | |
|---|---|
| DVFMKGLSMAKEGV | (SEQ ID NO:1) |
| VFMKGLSMAKEGV | (SEQ ID NO:2) |
| FMKGLSMAKEGV | (SEQ ID NO:3) |
| MKGLSMAKEGV | (SEQ ID NO:4) |
| KGLSMAKEGV | (SEQ ID NO:5) |
| GLSMAKEGV | (SEQ ID NO:6) |
| LSMAKEGV | (SEQ ID NO:7) |
| SMAKEGV | (SEQ ID NO:8) |
| MAKEGV | (SEQ ID NO:9) |
| AKEGV | (SEQ ID NO:10) |
| KEGV | (SEQ ID NO:11) |
| MDVFMKGLSMAKEG | (SEQ ID NO:12) |
| MDVFMKGLSMAKE | (SEQ ID NO:13) |
| MDVFMKGLSMAK | (SEQ ID NO:14) |
| MDVFMKGLSMA | (SEQ ID NO:15) |
| MDVFMKGLSM | (SEQ ID NO:16) |
| MDVFMKGLS | (SEQ ID NO:17) |
| MDVFMKGL | (SEQ ID NO:18) |
| MDVFMKG | (SEQ ID NO:19) |
| MDVFMK | (SEQ ID NO:20) |
| MDVFM | (SEQ ID NO:21) |
| MDVF | (SEQ ID NO:22) |
| DVFMKGLSMAKEG | (SEQ ID NO:23) |
| DVFMKGLSMAKE | (SEQ ID NO:24) |
| DVFMKGLSMAK | (SEQ ID NO:25) |
| DVFMKGLSMA | (SEQ ID NO:26) |
| DVFMKGLSM | (SEQ ID NO:27) |
| DVFMKGLS | (SEQ ID NO:28) |
| DVFMKGL | (SEQ ID NO:29) |
| DVFMKG | (SEQ ID NO:30) |
| DVFMK | (SEQ ID NO:31) |
| DVFM | (SEQ ID NO:32) |
| DVF | (SEQ ID NO:33) |
| GLSMAKEG | (SEQ ID NO:34) |
| GLSMAKE | (SEQ ID NO:35) |
| GLSMAK | (SEQ ID NO:36) |
| GLSMA | (SEQ ID NO:37) |
| GLSM | (SEQ ID NO:38) |
| GLS | (SEQ ID NO:39) |
| GL | (SEQ ID NO:40) |
| LSMAKEG | (SEQ ID NO:41) |
| LSMAKE | (SEQ ID NO:42) |
| LSMAK | (SEQ ID NO:43) |
| LSMA | (SEQ ID NO:44) |
| LSM | (SEQ ID NO:45) |
| LS | (SEQ ID NO:46) |

2. The peptide according to claim 1, whereby the individual components are L-amino acids.

3. The peptide according to claim 1, whereby the individual amino acids are D-amino acids.

4. The peptide according to claim 1, in which the amino acid proline is substituted in the N-terminal position.

5. The peptide according to claim 1, in which the amino acid proline is substituted in the C-terminal position.

6. The peptide according to claim 1, in which the amino acid proline is substituted in the N-terminal position and in the C-terminal position.

7. The peptide according to claim 1, which are acetylated in the N-terminal position.

8. The peptide according to claim 1, which are amidated in the C-terminal position.

9. The peptide according to claim 7, which are acetylated in the N-terminal position and amidated in the C-terminal position.

10. The peptide according to claim 1, characterized in that the amino acid valine (V) is replaced by the amino acid proline (P).

11. A pharmaceutical agent for use in the therapy of diseases in which the increased occurrence of free radicals plays a pathophysiological role, characterized by at least one peptide according to claim 1.

12. A pharmaceutical agent for use in the therapy of diseases with acute hypoxia or ischemia in an organ system of the body, in particular in the central nervous system, characterized by at least one peptide according to claim 1.

13. A pharmaceutical agent for use in the therapy of Recklinghausen-Appelbaum diseases, such as the Hallervorden-Spatz disease, characterized by at least one peptide according to claim 1.

14. A pharmaceutical agent for use in the therapy of neurodegenerative diseases, in particular Alzheimer's disease, the Lewy Body variant of Alzheimer's disease, Parkinson's disease, the multisystem atrophy, the Lewy Body dementia or Huntington's chorea, and all states similar to these neurodegenerative diseases, characterized by at least one peptide according to claim 1 as an active ingredient.

15. A pharmaceutical agent according to claim 11, which is prepared for oral administration.

16. A pharmaceutical agent according to claim 11, which is prepared for rectal administration.

17. A pharmaceutical agent according to claim 11, which is prepared for administration by inhalation.

18. A pharmaceutical agent according to claim 11, which is prepared for transdermal administration.

19. A pharmaceutical agent according to claim 11, which is prepared for transmucosal administration.

20. A pharmaceutical agent according to claim 11, which is prepared for administration via active ingredient-containing implants.

21. A pharmaceutical agent according to claim 11, which is prepared for intracerebroventricular administration.

22. A pharmaceutical agent according to claim 11, which is prepared for administration by injection.

23. A pharmaceutical agent according to claim 11, which is prepared for transnasal administration.

24. A pharmaceutical agent according to claim 11, which is prepared for administration by infusion.

25-26. (canceled)
